(19) United States
(12) Patent Application Publication
(10) Pub. No.: US 2026/0000380 A1
(43) Pub. Date: Jan. 1, 2026

(54) COMPUTER-ASSISTED SYSTEM AND METHOD OF HEART MURMER CLASSIFICATION

(71) Applicant: KARDIO DIAGNOSTIX INC., Nova Scotia (CA)

(72) Inventors: Robert Chen, Bedford (CA); Mohammed Shameer Iqbal, Middle Sackville (CA); Santokh Dhiloon, Halifax (CA)

(21) Appl. No.: 19/107,184

(22) PCT Filed: Sep. 1, 2023
(86) PCT No.: PCT/CA2023/051161

§ 371 Date: Feb. 27, 2025

(30) Foreign Application Priority Data
Sep. 1, 2022 (CA) 3171784

(51) Int. Cl. A61B 7/04; A61B 5/00

(52) U.S. Cl. CPC A61B 7/04; A61B 5/7257; A61B 5/7267

(57) ABSTRACT

A method to classify heart murmurs as benign or pathologic. A digitized acoustic heart signature of a patient is captured on a computing device and processed using Fast Fourier Transformation to identify a plurality of component frequency waveforms, each having a power value. Based on the waveforms' power values, they are classified into a primary frequency waveform, harmonic frequency waveforms, and non-harmonic frequency waveforms. The heart murmur of the patient is classified using a ratio of the power values of the harmonic waveforms as a portion of the composite power value of all the waveforms, and an interface indication is provided to the user of the computing device. A computing device and software program per the invention are also disclose.

This invention is in the field of cardiac diagnostic equipment and methodologies. More specifically, addresses the system and method for the rapid and streamlined diagnosis of benign versus pathologic heart murmurs in human patients.

### BACKGROUND

Mediated auscultation of the heart began in the 18th century with French physician Laennec's invention of the stethoscope. The diaphragm was added to the bell in the early 20th century. Digital stethoscopes available for the past 15 years record heart sounds conveniently but have not improved cardiac diagnosis by auscultation.

A cardiac murmur is the sound of blood flow through the heart and its vessels. The cardiac murmur presents a particular challenge for auscultation during childhood. About 60-90% of children have a cardiac murmur sometime during childhood (Coimbra, 2008) but only two percent (Chantepie, Soulé, Poinsot, Vaillant, & Lefort, 2016) are pathological or related to structural heart disease needing intervention; the remainder are benign from a cardiac perspective. 52% of adults have a cardiac murmur (Shub, 2003), from which many pathological murmurs of acquired structural heart disease must be identified.

Research shows the skill of cardiac auscultation amongst most physicians and other practitioners is poor despite diligent training in medical school (Mangione, 2001). Differentiating the murmurs related to structural heart disease from benign murmurs due to appropriate flow through the heart and its vessels is a conundrum. Missing pathological murmurs results in delay in diagnosis and management, sometimes resulting in fatal consequences, while misdiagnosing benign or innocent murmurs burdens the health care system with inappropriate referrals and expensive investigations (Danford, Nasir, & Gumbiner, 1993). Futility in training cardiac auscultation and growing demands on health care require an alternative to human assessment of cardiac sounds.

One of the primary areas in which technological advances have benefitted healthcare providers and patients alike has been the development of electronically assisted methods for assessing and diagnosing various medical conditions in patients, which would previously have been diagnosed or analyzed solely by human providers. By allowing electronic or electronically-assisted analysis of captured sensor data from a patient in fields with limited numbers of capable human medical resources, the rate and often the quality of diagnosis can be improved.

Historically, there has been a throughput limitation in the medical identification and classification of cardiac conditions, including heart murmurs. Mainly using the example of pediatric cardiology, a few doctors are adequately trained to diagnose and classify heart murmurs in patients. The limited availability of these doctors restricts rapid access to diagnostic services required to analyze and treat, heart murmurs efficiently. Heart murmurs in pediatric and other patients have been diagnosed primarily with a manual review of the acoustic cardiac signal from a patient using a stethoscope or other type of microphone. The healthcare provider must listen very carefully to identify conditions requiring further investigations or treatment. Replacing this reliance on human healthcare providers listening to heart sounds of patients as the initial screening before classification and treatment would significantly improve accessibility and timeliness of care by eliminating large numbers of unnecessary referrals to cardiologists and improving identification of pathology to refer appropriately.

Some attempts have been made to analyze and classify cardiac conditions in patients using computer software assistance.

Several artificial intelligence methods are available to identify murmurs, but simply identifying a murmur is not helpful because no many children have murmurs. Several attempts have also been published examining the efficacy of spectral analysis and artificial neural networks to identify pathologic murmurs. The downfall has been the requirement to input examples of every pathology to ensure the reliability of the system. Our approach simulates the approach of the human brain to recognize and generalize the findings of innocent murmurs, thereby defining all other murmurs as pathologic. Additionally, this methodology does not require an artificial neural network in the process.

U.S. Pat. No. 9,168,018 discloses a system and method for classifying a heart sound. That reference uses a technique in which a heart sound signal is preprocessed and then fed through a plurality of neural networks, each trained to identify various heart conditions. The equipment and complexity of the invention of the '018 patent limits its commercial utility because of the requirement for significant resources, cost and maintenance to operate multiple neural networks, likely in a WAN or cloud-based implementation. Developing an alternative would be preferred. Costs would be kept in check, and such technology's rapid deployment and use would be enhanced.

A software-assisted solution for rapidly classifying heart murmurs in human patients without a persistent WAN connection would be desirable to broaden the number of environments in which the technology could be deployed and used.

Providing a solution for early classification of heart murmurs requiring more detailed assessment or treatment without the need for an initial physician assessment or operation of the necessary equipment would be most desirable and find utility in certain verticals of the healthcare market.

### SUMMARY OF THE INVENTION

The disclosed invention presents a computer-implemented method and corresponding device that automates the classification of heart murmurs with improved accuracy. The method involves capturing digitized acoustic heart signatures of patients using a connected digital stethoscope. The captured heart signature undergoes signal processing using a fast Fourier transformation, revealing a range of component frequency waveforms in the heart sound.

For each component frequency waveform, the method calculates a power value representing the intensity of that waveform. The component frequency waveforms are categorized into three types: primary frequency waveform (the one with the highest power value), harmonic frequency waveforms (those sharing common factors with the primary waveform's frequency), and non-harmonic frequency waveforms (those not sharing factors with the primary waveform).

The invention calculates composite power values for all component frequency waveforms and separate power values for harmonic and non-harmonic frequency waveforms. The method accurately classifies the heart murmur by determining the harmonic ratio, representing the proportion of the harmonic power value to the composite power value. If the harmonic ratio exceeds a predefined benign threshold value, the murmur is classified as benign; otherwise, it is classified as pathologic.

The classification results are presented to the user through an interface display, which clearly indicates the heart murmur's classification. Additionally, the invention can store the captured heart signature and associated classified details for reference.

The critical difference between this approach and the prior art is its focus on understanding the acoustic signature of innocent murmurs. This signature is characterized by the human ear which performs a Fast Fourier Transform analyzed by the brain. The brain looks for the presence of resonance and the degree of resonance allows the classification of the murmur as innocent or not. The current method does not require knowing every pathology that exists but emphasizes the certainty of recognizing normal.

In a first embodiment, the invention comprises a computer-implemented method of classifying heart murmurs in patients wherein several steps are undertaken using a computing device hosting appropriate software to execute the method. The computing device will capture a digitized acoustic heart signature of the patient, being the captured heart signature.

The computing device can be many types of devices-it might comprise a standard personal computer hosting the necessary software of the method of the present invention, or in other cases, could also be a smart device or mobile device that was capable of the required connection to a digital stethoscope or other capture device. The present invention's software could also be installed on other pre-existing or purpose-built diagnostic hardware in medical environments. All such types of computing devices will be understood to those skilled in the art and insofar as they can connect to or capture from a connected device the necessary digital acoustic cardiac signature to be within the scope of this invention

In some cases, the digitized acoustic heart signature might be a previously captured and recorded file. In other cases, the present invention's software can accommodate the capture of the live acoustic heart signature of the patient via a connection to a digital stethoscope or other wearable or sensor device. It will be understood to those skilled in the art that the capture devices used in conjunction with the computing device could be of many types and all such devices capable of the measurement and capture, in conjunction with the computing device and hosted software thereon, of the necessary digital acoustic cardiac signature data are all contemplated within the scope of the present invention.

The next step of the method comprises a signal processing step in which the captured heart signature is processed with a Fast Fourier Transformation (FFT) to identify a plurality of component frequency waveforms thereof. For each component frequency waveform isolated in the FFT step, a power spectrogram is rendered to determine a power value of the component frequency waveform. The rendering of power spectrogram and measuring the amplitude or power value of a component frequency waveform will be understood by those skilled in the art.

Once the power value for each component frequency waveform is determined, the aggregate group of waveforms will be classified as:
a. “primary frequency or frequencies,” which will be referred to as “primary frequency” below, being the component frequency waveform with the most significant power value or values;
b. harmonic frequency waveforms, being any component frequency waveforms that factor with the same denominator as the primary frequency waveform; and
c. non-harmonic frequency waveforms, being any component frequency waveforms that do not factor with the same denominator as the primary frequency waveform;

Effectively in grouping and classifying the component frequency waveforms, the harmonic frequency waveforms can also be described as resonant with the primary frequency waveform. In contrast, the non-harmonic frequency waveforms are non-resonant with the primary frequency waveform. Effectively, the presence of a greater non-harmonic or non-resonant frequency distribution indicates the presence of a pathologic heart murmur. In contrast, the presence of a more significant harmonic or resonant frequency distribution is an indicator of a benign heart murmur.

Following the classification of the component frequency waveforms, the software will calculate a composite power value, being the total of the power values for all of the component frequency waveforms, along with a harmonic power value and a non-harmonic power value, being the total of the power values for all of the harmonic frequency waveforms and the total of the power values for all of the non-harmonic frequency waveforms, respectively.

In the next step of the method, the computer and software will classify the heart murmur of the patient by determining a harmonic ratio being the ratio of the harmonic power value as a portion of the composite power value and comparing the harmonic ratio to a defined benign threshold value. If the harmonic ratio is greater than the defined benign threshold value the heart murmur condition of the patient is classified as benign, and if the harmonic ratio does not reach the defined benign threshold value the heart murmur condition of the patient is classified as pathologic. It is anticipated that in most embodiments, the specified threshold value is the ratio of the non-harmonic power value as a portion of the composite power value. Still, it will be understood that other means of comparing the harmonic to the composite power value could also result in the same result and all such values or formulae for determination of the defined benign threshold value are contemplated within the scope of the present invention.

Following classification of the heart murmur of the patient as benign or pathologic, the system will provide an interface indication to the user of the computing device of the patient's benign or pathologic heart murmur classification.

In certain embodiments of the system and method of the invention the captured heart signature could be stored in memory along with the classified heart murmur details of the patient.

In addition to the method as disclosed, the present invention also comprises a device for use in a method of classifying heart murmurs in patients comprising in respect of a patient, said device comprising a computer with a processor, memory, data interface for capture of digitized acoustic heart signatures of patients, a human interface display and software operable thereon to execute the method.

Further embodiments of the invention comprise the software/processor instructions for use on a computer's processor comprising a processor, memory, data interface for capture of digitized acoustic heart signatures of patients, and a human interface display, for execution of the method.

It is explicitly contemplated that the scope of the present invention to which the Inventors are entitled to protection includes the method as outlined, as well as a hardware device hosting software which will permit the execution of various embodiments of the method and the software itself which is capable of executing the steps of the method as outlined in conjunction with the requisite hardware.

The present invention offers a more reliable, consistent, and objective method for classifying heart murmurs, reducing the subjectivity and potential errors associated with manual interpretation. This innovation empowers medical professionals with a valuable tool that aids in making accurate diagnoses, leading to improved patient care and better treatment outcomes. Furthermore, integrating computational technology enhances the efficiency and effectiveness of the classification process.

### DESCRIPTION OF THE DRAWINGS

To easily identify the discussion of any particular element or act, the most significant digit or digits in a reference number refer to the figure number in which that element is first introduced. The drawings enclosed are:

FIG. 1 is a flowchart showing the steps in one embodiment of the method of the present invention; and

FIGS. 2 through 4 are power spectrograms and signal analysis supporting diagrams used to demonstrate the present method.

### DETAILED DESCRIPTION OF ILLUSTRATED EMBODIMENTS

As outlined herein, the present invention comprises a computer-implemented method of classifying heart murmurs in patients, using streamlined methodology and hardware and software combinations.

It is desired to provide background and context around characteristics of various heart murmur technology to enhance the understanding of the value and utility of the present invention.

Laminar and turbulent flow refer to different fluid movement patterns within a vessel or conduit, such as blood flow within blood vessels. In cardiac flow, these terms describe how blood moves through the heart and its associated blood vessels.
1. Laminar Flow: Laminar flow is characterized by smooth, organized, and streamlined movement of fluid particles along well-defined paths within a vessel. In the case of blood flow, laminar flow occurs when blood moves in layers or laminae, with each layer of blood maintaining a consistent velocity and direction. The fluid particles in the vessel's center move faster than those near the vessel walls. Laminar flow is often associated with low velocities and is more common in larger blood vessels with consistent diameters.
2. Turbulent Flow: Turbulent flow, on the other hand, is characterized by chaotic, irregular, and often rapid movement of fluid particles within a vessel. It occurs when the flow velocity exceeds a certain threshold, causing disturbances in the fluid dynamics. These disturbances lead to the mixing of fluid layers, eddies, and vortices, resulting in an unpredictable flow pattern. Turbulent flow is more likely to occur in situations where blood flow is impeded, such as in areas of vessel narrowing (stenosis) or in regions where blood vessels bifurcate or branch.

In the context of cardiac flow, both laminar and turbulent flow patterns can have clinical significance. Laminar flow is usually the desired state, as it allows for efficient and smooth blood movement through the cardiovascular system. Turbulent flow, on the other hand, can indicate underlying issues such as arterial plaques, valve problems, or other obstructions That Disrupt the Normal Flow of Blood

### Characteristics of Murmurs Due to Laminar Flow

Laminar flow creates tissue vibration heard as primary frequency. Tissues of adjacent structures are stimulated to vibrate at frequencies harmonically related to this primary frequency. Analogously, different musical string instruments playing the same note sound different or have different voices because of these harmonic differences-the instruments are distinguished not by the primary frequency but rather by the harmonic frequencies accompanying it. These sounds are also described as resonant.

### Characteristics of Murmurs Caused by Turbulent Flow

Turbulent flow occurs when the velocity of a fluid is so high its Reynolds Number is exceeded, and organized laminar flow degrades into multiple jets with random vectors. Each jet produces a primary frequency and accompanying harmonic frequencies. These multiple primary frequencies are not harmonically related, resulting in dissonant sounds.

Resonance and dissonance describe sound quality in the parlance of cardiac auscultation or tambour in music. Recognizing dissonance is essential to identifying pathological murmurs. Few dissonant murmurs are benign and rarely are resonant murmurs pathological because normal blood flow in the circulation is laminar.

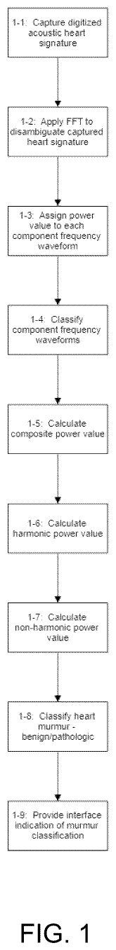

Resonant sounds can be identified using fast Fourier transformation of the murmur signal. Fast Fourier transformation separates the murmur waveform into its component sine waves. The largest amplitude wavelength will be defined as the primary frequency. All other frequencies found in the Fourier transform that factor with a common denominator as the primary frequency will be considered harmonic frequencies. The remainder will be considered dissonant frequencies. The proportion of resonant to dissonant frequencies will be used to identify benign murmurs. FIG. 1 shows our preliminary analysis of a limited number of heart sounds. Note we can extract the harmonic frequencies from the phonocardiogram. The power or intensity of the graphs reveal how it can segregate predominantly resonant frequencies from others in the phonocardiogram revealing the innocent murmurs.

The critical difference between this approach and all the others is the focus on understanding the acoustic signature of innocent murmurs. This signature is characterized by the human ear which performs a fast Fourier transform that is analyzed by the brain. The brain looks for the presence of resonance and the degree of resonance allows the classification of the murmur as innocent or not. This process does not require knowing every pathology that exists but emphasizes the certainty of recognizing normal.

### Prior Art and Methodology of Murmur Classification

In traditional prior art methods of grouping and classifying heart murmurs in patients, cardiologists will typically identify a murmur in a patient by listening to a stethoscope or other audio signal. Manual audible processing by the individual results in significant limitations and availability of this service, since their only small numbers of trained and experienced cardiology professionals who can provide this service. This results in significant throughput limitations in the medical system in terms of the ability of the system to provide sufficient diagnostic services of this nature to all the younger or older individuals who might require this type of diagnosis.

In the manual processing or identification of a heart murmur, the cardiologist is listening for dissonant or non-harmonic turbulent flow in the acoustic signature. If the acoustic signature of the patient has significant non-harmonic components to it, then the heart murmur requires further diagnosis because it is pathologic.

Other prior art methods have included the use of ECGs or other sophisticated equipment to assist in diagnosing or identifying pathologic cardiac murmurs. These again require specialized staff who know how to operate them and read the results and require expensive equipment to be used. The ECG assisted methods as well as other computer-based methods requiring significant data capture to be processed through one or more artificial intelligence networks to identify cardiac murmurs of pathologic nature or other types of cardiac conditions are all, along with the traditional means of human/manual audible signal processing by a cardiologist simply listening to the heart signature of the patient, all represent prior art and rate limited methods for diagnosis of this nature. Looking back to the manual or human executed prior art method, namely that of audibly listening to the cardiac acoustic signature of the patient, it is believed that finding a way to automate the streamlined audible processing of the cardiac acoustic signature of patient would be the approach which could be attempted in the present situation to provide a machine-assisted method of rapid assessment and identification of benign versus pathologic cardiac murmurs.

### Deconstructing the Captured Heart Signature

The cardiac signature of the patient can be captured using a digital stethoscope or other similar equipment, for processing on a computer in accordance with the remainder of the present invention. It is explicitly contemplated that the system and method of the present invention will be able to be practiced with widely available and cost-effective equipment even as simple as a smart phone or other mobile device with the necessary software installed thereon and with an operative connection to a digital stethoscope. The types of equipment which can be used are described in further detail throughout the remainder of the specification.

Upon the capture of the heart signature of the patient, as a digital file or digital sample, the sample will be disambiguated, or parsed into its component frequencies. Fourier analysis converts a signal from its original domain of for example time or space, to a representation in the frequency domain. A discrete Fourier transform is processed by decomposition of a sequence of values into component frequencies. This type of an operation is useful across many fields, but a regular transform of this nature is often too slow to be commercially practical.

A fast Fourier transform is an algorithm that computes the discrete Fourier transform of a sequence, or the inverse thereof, and sufficient speed to be useful-the details of an FFT algorithm will be understood to those skilled in the art and are all contemplated within the scope of the present invention—this type of a mathematical procedure reduces the complexity of the calculation and computation of the discrete Fourier transform. FFT operations are widely used in sound processing applications amongst others-the importance of this type of an operation in digital sound sampling and the like is that it has made work in the frequency domain computationally feasible, in fields including filtering algorithms as well as fast algorithms for discrete cosine or sine transformations. The difference in speed can be significant, especially in longer datasets. In many cases, FFT algorithms are more accurate than directly evaluating a DFT definition.

Effectively in grouping and classifying the component frequency waveforms, the harmonic frequency waveforms can also be described as resonant with the primary frequency waveform. In contrast, the non-harmonic frequency waveforms are non-resonant with the primary frequency waveform. Effectively the presence of a greater non-harmonic/non-resonant frequency distribution is an indicator of the presence of a pathologic heart murmur. In contrast, the presence of a greater harmonic/resonant frequency distribution is an indicator of a benign heart signature or murmur.

Upon applying an FFT algorithm of the nature outlined above and as will be understood to those skilled in the art of digital signal processing, the captured heart signature of the patient will be separated into a plurality of component frequency waveforms. These are effectively all of the different sound components making up the entirety of the sound within the captured data sample of the captured heart signature of the patient. The plurality of component frequency waveforms will be further processed and used in the remainder of the method of the present invention.

### Method Overview

Referring to FIG. 1 there is shown a flow chart of one embodiment of the steps of a method in accordance with the present invention conducted using a computing device hosting software capable of executing the necessary steps of the method of the present invention.

In the first step of the method of the present invention, shown at step 1-1, the computing device will capture a digitized acoustic heart signature of the patient, which is the captured heart signature. As outlined throughout, the digitized acoustic heart signature could be captured by the computing device from and operatively connected digital stethoscope or other type of a sensor or device capable of capturing the necessary information for a digitized sample of the acoustic heart signature of the patient to be captured. The captured heart signature could be stored in permanent memory of the computing device or simply stored in volatile onboard memory to execute the remainder of the method's steps and subsequently purged therefrom.

In the second step of the method, a signal processing step 1-2 is executed. The signal processing step comprises processing the captured heart signature/digital sample using a fast Fourier transformation to identify and disambiguate a plurality of component frequency waveforms thereof.

Each of the component frequency waveforms will then be assessed to determine a power value of that component frequency waveform which will be assigned in respect thereof for the remainder of the classification method of the present invention. The power value of the component frequency waveform would likely be assessed by using the computing device and software instructions to render a power spectrogram for the component frequency waveform and assess the power value on that basis. Determination of the power values of the component frequency waveforms is shown at step 1-3.

Following the determination of the associated power values, the component frequency waveforms will be classified, shown at Step 1-4. A primary frequency waveform, which is the component frequency waveform with the largest power value or amplitude, will be identified. Identification of the primary frequency waveform based upon amplitude or power values will again be easily understood to those skilled in the art of digital signal processing and any specific mathematics involved for identification of this waveform on this basis will be understood to be within the scope of the present invention. Each component frequency waveform other than the primary frequency waveform will be classified into two categories of harmonic or no harm frequency waveforms. Harmonic frequency waveforms are any component frequency waveforms that mathematically factor with the same denominator as the primary frequency waveform. Non-harmonic frequency waveforms are any component frequency waveforms that do not factor with the same denominator as the primary frequency waveform, which in a sound context indicates that they are non-harmonic dissonant sound waves which would be detected by a cardiologist in the traditional manual and audible diagnosis method as indicating turbulent flow in the cardiac signature of the patient.

Following the classification of each of the component frequency waveforms in step 1-4, a composite power value will be calculated, being the total of the power values for all of the component frequency waveforms (Step 1-5), the harmonic power value will be calculated which is the total of the power values for all of the harmonic frequency waveforms (Step 1-6), and a non-harmonic power value will be calculated which is the total of the power values for all of the non-harm frequency waveforms (Step 1-7).

Based on the calculation of all of these variables, at step 1-8 the computing device can classify the heart murmur of the patient by determining a harmonic ratio which is the ratio of the harmonic power value as a portion of the compass at power value. If the harmonic ratio exceeds a defined benign threshold value, the heart murmur condition of the patient is classified as benign, and if the harmonic ratio does not exceed or reach the defined benign threshold value, indicating dissonance in the heart signature, the heart murmur condition of the patient is classified as pathologic.

At Step 1-9, the computing device will provide an interface indication to its user of the benign or pathologic heart murmur classification of the patient.

In most cases, the defined threshold value is the ratio of the non-harmonic power value as

a portion of the call opposite power value. Effectively, a higher harmonic ratio than the non-harmonic ratio indicates benign heart murmur classification whereas a higher non-harmonic ration indicates a pathologic heart murmur classification requiring further attention. It will be understood that other mathematics can also be used to select or define the defined benign threshold value of the method of the present invention and all such approaches are contemplated within the scope of the present invention.

In certain embodiments of the method of the present invention, the captured heart signature and the patient's classified heart murmur details could be stored in the memory of or operatively connected to the computing device for archival purposes.

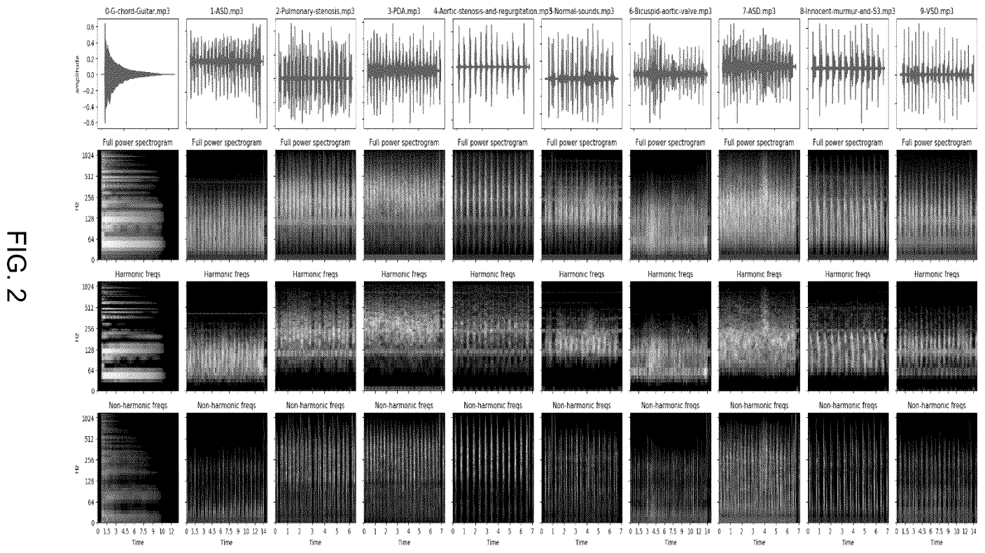

FIGS. 2 through 4 are provided to further demonstrate in conjunction with the description. Referring to FIG. 2 there is shown a phonocardiogram in the top row thereof, and a full power spectrogram of a fast Fourier transform of the phonocardiogram.

The third row of FIG. 2 shows the harmonic frequencies extracted from analysis of the FFT and the power value for each of the harmonic frequencies displayed as a power spectrogram. The fourth/bottom row of this Figure shows the non-harmonic frequencies extracted from analysis of the FFT and the power value for each non-harmonic frequency displayed as a power spectrogram.

For demonstrative purposes, the first column of FIG. 2 is a spectrogram. Note the similarity of the full power spectrogram to the harmonic frequency power spectrogram that suggests the guitar produces primarily harmonic frequencies making the sound resonant. Similarly, the ASD, innocent murmur is primarily resonant. However, pulmonary stenosis, aortic stenosis, and VSD show the harmonic spectrogram is much less like the full spectrogram indicating less resonant sound.

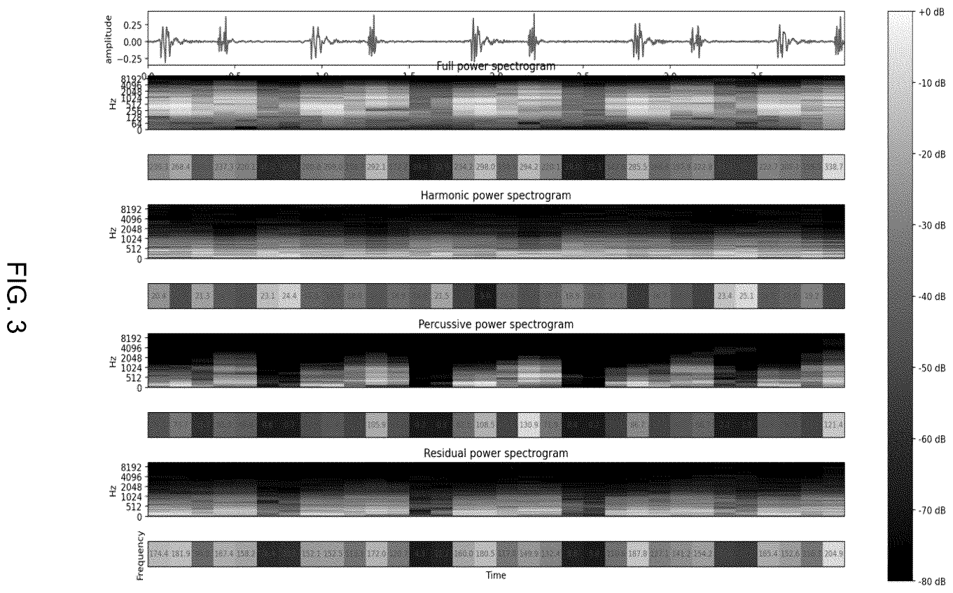

To illustrate this further, referring to FIG. 3 we applied the same signal processing methodology of the present invention on an innocent murmur where there is an “extra” murmur in between cardiac cycles. However, when we apply the method of the present invention, we can see there is more energy in the harmonic bucket compared to residual ones—indicating a benign murmur.

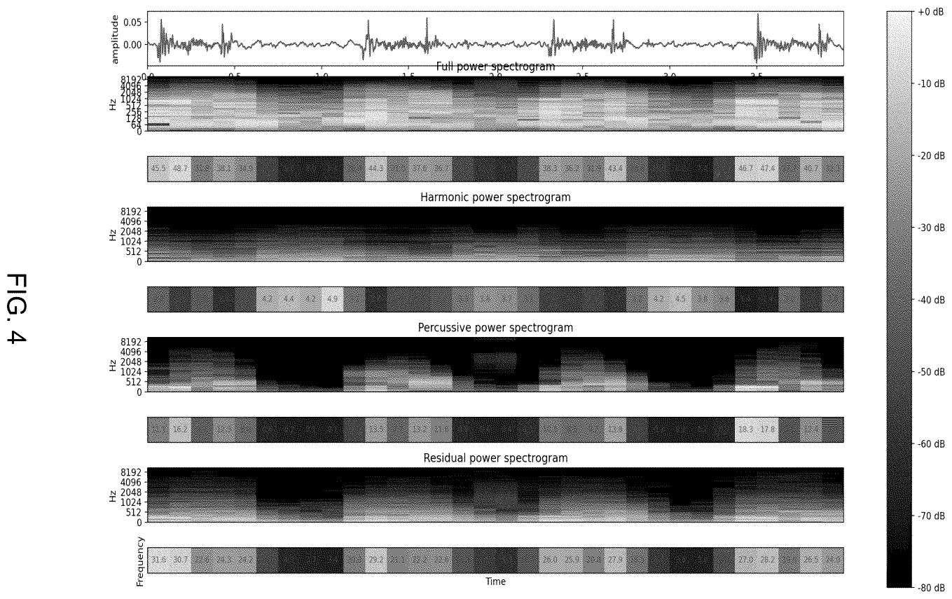

By contrast as shown in FIG. 4, we applied the same signal processing methodology of the present invention on a sample of a VSD murmur (pathological). In this case, there is more energy in the residual and the non-harmonic bucket.

### Interface Display

As outlined in the claims and throughout this document, the ultimate goal of the method of the present invention is, upon the classification of the heart murmur in a patient has been nine or pathologic, to provide a basic interface indication on a human interface operatively connected to the computing device of the present invention, permitting either the patient or the doctor or health care provider operating same to have a first stage indication of the classification of the heart murmur in the patient. At that point, if a pathologic heart murmur was indicated, additional diagnostics could be conducted. Any type of an interface display, from simple to complicated, can be contemplated and understood to those skilled in the art and all are contemplated within the scope of the present invention.

### Computing Device

As has been outlined throughout, the computing device on which the method of the present invention could be practiced could comprise many different types of devices, with attendant modifications made to the software component of the present invention for execution thereon. The computing device could for example be a personal computer or a device of that nature, or a portable electronic device such as a smart phone, tablet computer and the like. Any type of a computing device capable of hosting a software component for the execution of the method of the present invention and having a connection or a bus permitting communication thereof with a digital stethoscope or other means of capture of the digital heart signature of the patient are all contemplated within the scope of the present invention.

The system and method of the present invention could also be practiced by the installation of the software component executing the method outlined herein on preexisting specific medical diagnostic hardware capable of capturing the necessary digital cardiac audio signature file or sample. Installation of software permitting the execution of the present invention on such pre-existing medical hardware will also be understood by those skilled in the art to be within the scope of the present invention.

In addition to a processor and memory it will be understood to those skilled in the art as key components of the computing device of the present invention, the computing device would also include the necessary boss or connection/interface to permit communication of the computing device with a cardiac signature capture device such as a digital stethoscope or the like. The computing device will also include necessary human interface components such as a screen of the like by which results of heart murmur classifications conducted in accordance with the method of the present invention could be displayed to the user, as well as a keyboard, touchscreen interface of the like permitting the selection of parameters or the execution of the method.

In the ideal scenario, the computing device of the present invention would be a mobile computing device, usable by doctors in multiple locations or easily transportable between diagnostic locations and the like.

### Software Component

The computing device of the present invention will host or store within memory a software component for the execution of the method the present invention in communication with the additional necessary components of the computing device and the cardiac signature capture hardware. For example, where a mobile device or a smart device or the like was the actual computing device used to execute the method, a software app for installation on that type of the device will be understood to those skilled in the art. Similarly, if the computing device to be used is a desktop computer of the like, the software component could be prepared in a programming language or in the necessary fashion to be hosted or stored within the memory of that type of that type of the device for execution on the processor and within the memory thereof.

It will be apparent to those of skill in the art that the present invention can be optimized for use in a wide range of conditions and application by routine modification. It will also be obvious to those of skill in the art that there are various ways and designs with which to produce the apparatus and methods of the present invention. The illustrated embodiments are therefore not intended to limit the scope of the invention, but to provide examples of the apparatus and method to enable those of skill in the art to appreciate the inventive concept.

Those skilled in the art will recognize that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. The terms “comprise” and “comprising” should be interpreted as referring to elements, components, or steps in a non-exclusive manner, indicating that the referenced elements, components, or steps may be present, or utilized, or combined with other elements, components, or steps not expressly referenced.

1. A computer-implemented method of classifying heart murmurs in patients comprising in respect of a patient, using a computing device and a cardiac classification software component operating thereon:
a. capturing a digitized acoustic heart signature of the patient, being the captured heart signature;
b. in a signal processing step, processing the captured heart signature using a fast Fourier transformation to identify a plurality of component frequency waveforms thereof;
c. for each component frequency waveform, determining a power value of the component frequency waveform;
d. classifying the component frequency waveforms as:
i. a primary frequency waveform, being the component frequency waveform with the largest power value;
ii. harmonic frequency waveforms, being any component frequency waveforms that factor with the same denominator as the primary frequency waveform; and
iii. non-harmonic frequency waveforms, being any component frequency waveforms that do not factor with the same denominator as the primary frequency waveform;
e. calculating:
i. a composite power value, being the total of the power values for all of the component frequency waveforms;
ii. a harmonic power value, being the total of the power values for all of the harmonic frequency waveforms; and
iii. a non-harmonic power value, being the total of the power values for all of the non-harmonic frequency waveforms; and
f. classifying the heart murmur of the patient by determining a harmonic ratio being the ratio of the harmonic power value as a portion of the composite power value, wherein:
i. if the harmonic ratio exceeds a defined benign threshold value the heart murmur condition of the patient is classified as benign; and
ii. if the harmonic ratio does not reach the defined benign threshold value the heart murmur condition of the patient is classified as pathologic; and
g. in an interface display step, providing an interface indication to the user of the computing device of the benign or pathologic heart murmur classification of the patient.

2. The method of claim 1 wherein the power value of each component frequency waveform is determined by rendering a power spectrograph of said component frequency waveform as isolated from the captured heart signature and calculating the power value based on the rendered power spectrograph.

3. The method of claim 1 wherein the defined threshold value is the ratio of the non-harmonic power value as a portion of the composite power value.

4. The method of claim 1 further comprising storing the captured heart signature along with the classified heart murmur details of the patient to memory associated with the computing device.

5. The method of claim 1 wherein the computing device captures the digitized acoustic heart signature of the patient from a connected digital stethoscope.

6. The method of claim 1 wherein the computing device comprises either a standard personal computer or a portable smart device of a user.

7. A device for use in a method of classifying heart murmurs in patients comprising in respect of a patient, said device comprising a computer with a processor, memory, data interface for capture of digitized acoustic heart signatures of patients, a human interface display and software operable thereon to execute the method of any one of claims 1 to 3.

8. The device of claim 7 wherein the device comprises either a standard personal computer or a portable smart device of a user.

9. The device of claim 7 further comprising a digital stethoscope connected to the data interface.

10. A computing device for use in a method of classifying heart murmurs in patients, said computing device comprising a processor, memory, a human interface device capable of providing data and interactions with a user, connected cardiac capture hardware to capture a digitized heart signature of a patient and processor instructions comprising a cardiac classification software component for executing the steps of a cardiac classification method, wherein the software application will, in operation on the computing device, execute a cardiac classification method comprising:
a. capturing data representing a digitized acoustic heart signature of the patient, being the captured heart signature;
b. in a signal processing step, process the captured heart signature using a fast Fourier transformation to identify a plurality of component frequency waveforms thereof;
c. for each component frequency waveform, determining a power value of the component frequency waveform;
d. classifying the component frequency waveforms as:
i. a primary frequency waveform, being the component frequency waveform with the largest power value;
ii. harmonic frequency waveforms, being any component frequency waveforms that factor with the same denominator as the primary frequency waveform; and
iii. non-harmonic frequency waveforms, being any component frequency waveforms that do not factor with the same denominator as the primary frequency waveform;
e. calculating:
i. a composite power value, being the total of the power values for all of the component frequency waveforms;
ii. a harmonic power value, being the total of the power values for all of the harmonic frequency waveforms; and
iii. a non-harmonic power value, being the total of the power values for all of the non-harmonic frequency waveforms; and
f. classifying the heart murmur of the patient by determining a harmonic ratio being the ratio of the harmonic power value as a portion of the composite power value, wherein:
i. if the harmonic ratio exceeds a defined benign threshold value the heart murmur condition of the patient is classified as benign; and
ii. if the harmonic ratio does not reach the defined benign threshold value the heart murmur condition of the patient is classified as pathologic; and
g. in an interface display step, providing an interface indication to the user of the computing device of the benign or pathologic heart murmur classification of the patient.

11. The computing device of claim 10 wherein the power value of each component frequency waveform is determined by rendering a power spectrograph of said component frequency waveform as isolated from the captured heart signature and calculating the power value based on the rendered power spectrograph.

12. The computing device of claim 10 wherein the defined threshold value is the ratio of the non-harmonic power value as a portion of the composite power value.

13. The computing device of claim 10 wherein the interface display step further comprises storing the captured heart signature along with the classified heart murmur details of the patient to memory associated with the computing device.

14. The computing device of claim 10 wherein the connected cardiac capture hardware is a digital stethoscope.

15. The cardiac classification software component for executing the steps of the cardiac classification method of claim 1.
